(19) United States
(12) Patent Application Publication
(10) Pub. No.: US 2003/0015225 A1
(43) Pub. Date: Jan. 23, 2003

(54) Apparatus for treating an article

(76) Inventor: Paul Stephen Nightingale, Exmouth (GB)

Correspondence Address: Ira S. Dorman, Suite 200, 330 Roberts Street, East Hartford, CT, 06108, US

(21) Appl. No.: 09/911,104

(22) Filed: Jul. 23, 2001

(51) Int. Cl. B08B 3/10

(52) U.S. Cl. 134/37; 15/310

(57) ABSTRACT

The apparatus comprises a flexible inflatable envelope 2 for receiving an article 1 and an inflation unit 12 comprising blower means for inflating said envelope. A first air-conducting passage 30 leads from said inflation unit 12 to said envelope 2, and a second passage 31 leads from said envelope 2 to said inflation unit 12 whereby air may be recirculated between the envelope and the inflation unit.

### TECHNICAL FIELD OF THE INVENTION

This invention relates to apparatus which can be used to treat an article such as (by way of example only) an item of furniture.

### BACKGROUND

One application of the present invention relates to the destruction of pests such as house dust mites, fleas, and silverfish. Another closely related aspect of the invention concerns allergenic material which is frequently present in the living environment, sometimes coming from an external source but often being associated with the presence of pests.

Proteins which are present in the droppings of house dust mites are a common form of allergenic material. When the droppings build up to a level which exceeds the tolerance of an individual, various forms of allergic reaction can be produced, some of which can be extremely serious.

Various forms of treatment have been proposed. A common suggested treatment is to regularly wash bedding and other items to remove the mites and their droppings. Although washing does effectively remove the water soluble allergens, modern washing machines are often designed to operate at a maximum temperature of 40° C., which is insufficient to kill the mites so that they will soon multiply to provide a new source of allergenic material. Freezing is sometimes successfully used to kill the mites, but this does not remove the mites nor does it denature any allergenic material which is present. It should also be noted that neither of these processes is suitable for treating large items such as mattresses or many soft furnishings.

GB 2 280 851-A describes the use of steam to destroy house dust mites and render their allergenic material ineffective. Similarly, in WO 96/03870 the house dust mite and its allergens are treated by injecting steam into an article followed by hot air drying.

GB 2 283 174-A discloses a form of treatment in which an article of furniture is placed in a heat-resistant bag which is vented to atmosphere. Hot air is blown through the bag to heat the article and kill any pests therein.

It is also known that various chemical treatments can be used to treat house dust mites and similar pests. For example, EP 0 168 243-A discloses the use of a tannic acid solution to treat house dust mites and their allergens, and GB 2 300 122-A discloses their control by coating surfaces with polysaccharide binding materials.

Heat treatment is also known to be a safe and effective way of killing a wide range of viruses and bacteria which may be harmful to human and animal health. A further aspect of the invention is concerned with the treatment of timber-containing articles e.g. to offer protection against rot, insect attack or to impart flame or fire-resistance. For example, it is well known that organo-boron products such as trimethyl borate will hydrolyse with moisture in wood to form boric acid, which acts as an excellent preservative and fire retardant.

The present invention seeks to provide apparatus which can be used to treat an article for various purposes, including (but not limited to) those mentioned above, and which is patentably different from any known form of apparatus.

### SUMMARY OF THE INVENTION

The present invention proposes apparatus for treating an article, comprising:

a flexible inflatable envelope for receiving said article;

an inflation unit comprising blower means for inflating said envelope;

a first air-conducting passage leading from said inflation unit to said envelope; and

a second passage leading from said envelope to said inflation unit whereby air may be recirculated between the envelope and the inflation unit.

By recirculating air through the envelope it is ensured that a stable environment can be maintained within the envelope during treatment of the article contained therein.

The inflation unit preferably comprises heating means for heating air supplied to the envelope. Due to the recirculation of air an accurate treatment temperature can be maintained within the envelope with minimum heat input.

The apparatus preferably further comprises a fluid reservoir (for a liquid or a gas) and means for introducing said fluid into the air flow to the envelope. Where the fluid is a liquid it is preferably introduced into the heating means such that the liquid is vaporised before being supplied to the envelope.

The cross sectional area of the first passage is preferably greater than that of the second passage. Further, a balance vent may be included to allow a small amount of external air to be added to the air entering the envelope through the first passage. This ensures that a positive inflation pressure is maintained within the envelope relative to the external environment and allows the envelope to re-inflate if it should be opened for inspection purposes.

### BRIEF DESCRIPTION OF THE DRAWINGS

The following description and the accompanying drawings referred to therein are included by way of non-limiting example in order to illustrate how the invention may be put into practice. In the drawings:

FIG. 1 is a general diagrammatic illustration of treatment apparatus in accordance with the invention,

FIG. 2 is a block diagram of the inflation unit of the apparatus

FIG. 3 is a detail of the unit of FIG. 3 showing a modification thereto, and

FIG. 4 is a similar general view to FIG. 1 but including a further modification to the treatment apparatus.

### DETAILED DESCRIPTION OF THE DRAWINGS

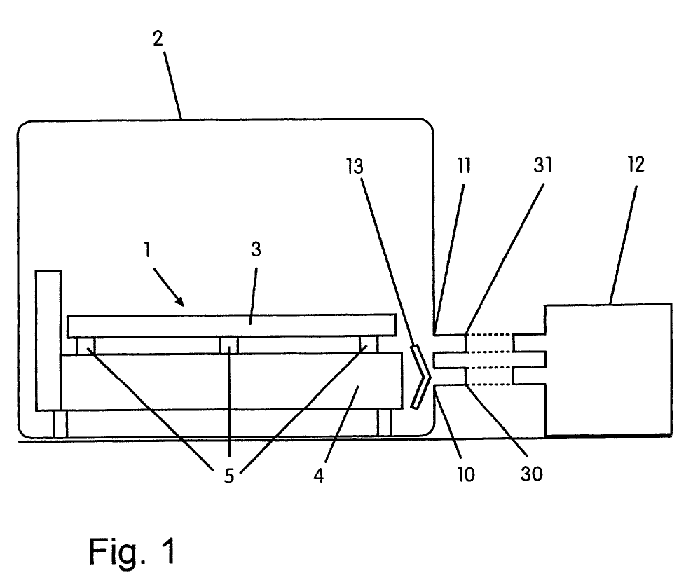

Referring firstly to FIG. 1, the apparatus comprises a flexible and inflatable envelope 2 which is preferably sealed but need not be completely impermeable. The envelope may have a zip fastener (not shown) or similar closure through which an item of furniture such as a bed 1 can be placed into the envelope prior to inflation. A preferred form of envelope comprises a layer of fibrous heat-insulation material sandwiched between two sheets of silicone coated polyamide fabric or similar flexible heat-resistant material.

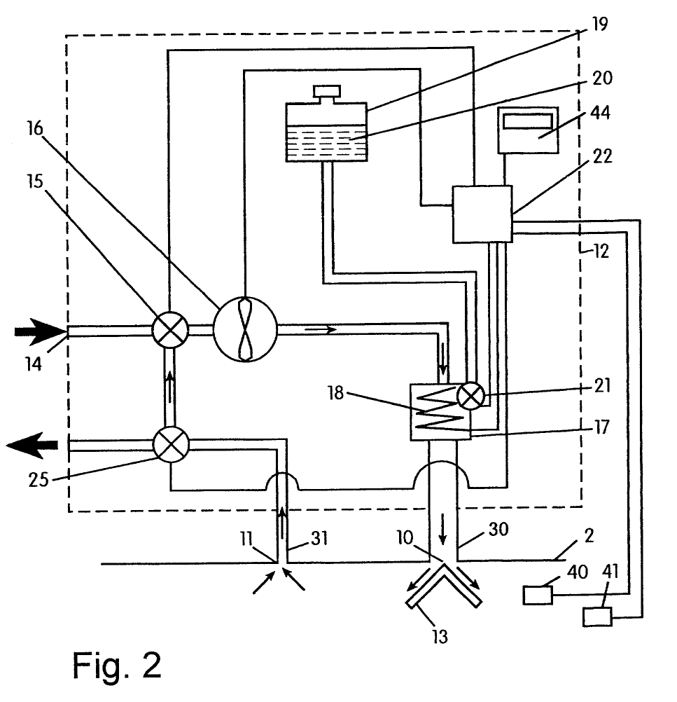

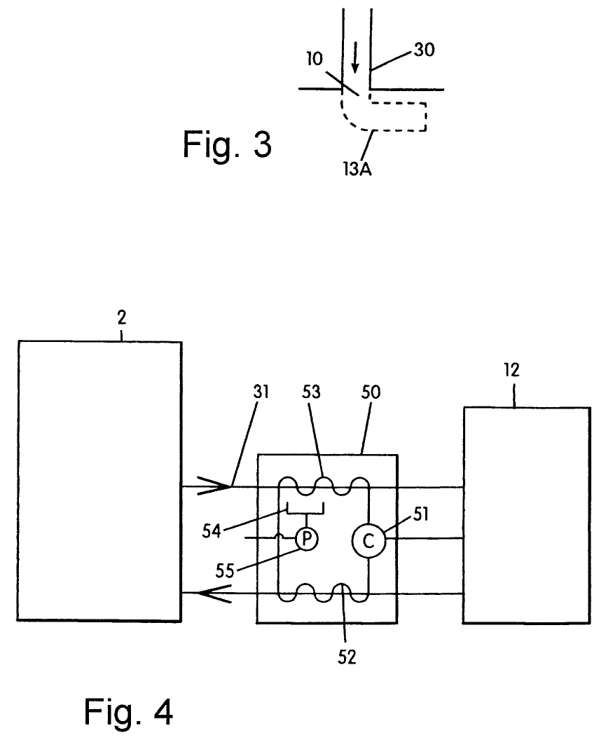

The envelope is connected with an inlet port 10 and an outlet port 11 which are connected by respective flexible ducts 30 and 31 to an inflation and treatment machine 12, described below. A baffle 13 is mounted over the inlet port 10 inside the envelope 2 (also shown in FIG. 2) to prevent a concentrated stream of hot air from being directed onto the article 1. Instead of a baffle the inlet port 10 could be provided with an angled inlet nozzle 13A, as shown in FIG. 3.

FIG. 2 shows the machine 12 in more detail. The machine comprises an inlet 14 for external air connected via a valve 15 to a blower 16. The outlet of the blower feeds air via a heater box 17 and duct 30 into the envelope 2. In the present example the heater box 17 contains at least one electrical heating element 18. It will be appreciated however that the heater box could instead incorporate gas or oil burners for heating the air passing therethrough. The valve 15 can be switched so that the blower draws air out of the envelope 2 via the duct 31 and a venting valve 25 to recirculate the air back to the envelope via the heater 17. The valve 15 is designed to permit the recirculated air to be mixed with about 5% ambient air to maintain inflation of the envelope 2. It will also be noted in FIG. 2 that the cross sectional area of the duct 30 supplying air to the envelope is significantly greater than that of the return duct 31 so that a positive inflation pressure is always maintained within the envelope 2.

The purpose of the valve 25 is to permit air to pass out of the envelope 2 to atmosphere when deflating the envelope at the end of a treatment cycle for example. It will be appreciated however that the valve 15 could be constructed to perform both functions, or evacuation could simply be achieved by uncoupling the return duct 31.

The machine 12 incorporates a dosing reservoir 19 for holding a liquid 20, which can be fed directly into the heater box 17 via a valve 21. Whilst gravity feed may be suitable for many applications it will often be preferred to use a dosing pump so that higher delivery rates and more accurate control can be achieved. In addition, it will often be preferable to spray the liquid into the heater box through a nozzle so that atomisation of the liquid is achieved to aid heating and vaporisation of the liquid. By introducing the liquid directly into the heater box 17 it can be ensured that the liquid is immediately and completely vaporised before entering the envelope 2 via the duct 30.

In some circumstances it may be desired to introduce a gas into the envelope 2, for which purpose the machine could include a gas reservoir arranged to feed gas at any convenient point into the recirculating air path.

The valves 15, 25 and 21, the blower 16 and the heating element 18 are controlled by a microcontroller 22 or other form of programmable sequential switching device. The microcontroller also receives input from at least one temperature sensor 40 (e.g. a thermocouple) and a humidity sensor 41, which are located within the envelope 2.

The microcontroller 22 will be required to apply different control sequences depending upon the use to which the apparatus is put. This is conveniently achieved by means of a smart card reader 44 which allows the user to change the operating program simply be inserting the appropriate card. If desired, the cards can be of the single use type for increased security. Alternatively, manual selection of the operating program can be provided, e.g. by incorporating a keypad.

The following examples will serve to illustrate the versatility of the apparatus and the wide range of uses to which it can be put.

### Heat Treatment

As discussed above, heat treatment of an article can be used to kill pests, bacteria or viruses and to denature certain proteinaceous allergens. By way of example, the following description relates specifically to treatment for house dust mites.

Referring to FIG. 1, a bed 1 or other article containing house dust mites and their droppings (e.g. a chair or settee) is placed inside the envelope 2. Any separable parts of the article such as the mattress 3 and base 4 are separated using spacers 5.

During initial inflation of the envelope 2 the valve 15 is switched so that the blower 16 feeds atmospheric air into the envelope from the inlet 14 via the duct 30. The heating element is not supplied with electrical power at this stage so that no heating of the air takes place. There then follows an initial pre-heating phase, during which the temperature inside the envelope 2 is increased to about 50° C. to enhance the vapour-holding properties of the air. This is achieved by switching the valve 15 to recirculate air from the envelope via the heater box 17 with the heating element/s supplied with electrical power. The pre-heating phase is relatively short (e.g. 5 minutes), being of sufficient length to heat the air in the envelope but of sufficiently short duration for the article to remain relatively cool.

At the end of the pre-heating phase the valve 21 is opened or the dosing pump operated to feed liquid into the heater box 17 during air recirculation, the heating element/s being switched to a heating level which is sufficient to cause the liquid to be immediately and fully vaporised. The vapour passes into the envelope over a period of 15 to 20 minutes to saturate the air contained therein. The liquid reservoir may contain ordinary tap water, although the same means may be used to introduce liquid chemicals, poisons etc. For dust mite treatment the reservoir could be charged with an aqueous solution containing 2 to 4% tannic acid by volume (a protein denaturant), and 1 to 3% detergent or other wetting agent, by volume. The wetting agent further improves the efficiency by allowing the liquid to thoroughly wet and penetrate the article. When the vapour comes into contact with the cooler article it tends to condense out on the article so that at the end of this period the article is thoroughly wetted and may be saturated with liquid depending on the volume of liquid supplied.

The injection of vapour by valve 21 is controlled by the microcontroller 22 in order to maintain a constant level of humidity within the envelope 2, as detected by the humidity sensor 41.

The vapour-containing air inside the envelope is then subjected to a high level heating phase. During this phase the air is rapidly circulated via the blower 16, with the blower operating at maximum speed and the heating element/s 18 operating at maximum temperature, subject to thermostatic control to prevent overheating and damage to the apparatus or the article being treated. A sustained temperature as low as about 60° C. will kill many pests such as house dust mites, but to denature many allergenic proteins a temperature of about 75° C. must be sustained for several hours. It is generally preferred to use a temperature of at least 100° C., which will denature most allergenic proteins in a much shorter period. In order to ensure effective denaturing of many allergenic proteins the heating phase is preferably sustained for at least 30 minutes. The sustained temperature also allows the article to dry out during this phase.

During the parts of the treatment cycle where air is recirculated between the envelope 2 and the machine 12 it is desirable to draw a small amount of external ambient air into the recirculation path, preferably in the area of the area of the blower 16. This could be achieved by opening the valve 15 to the inlet 14, or by use of a small auxiliary vent. This ensures that a small positive pressure is maintained within the envelope 2 relative to external air pressure to ensure that the envelope remains fully inflated to compensate for any leakage and permit re-inflation if the envelope is opened for inspection during the treatment process.

Finally, the heating element 18 is switched off and the valve 15 is switched to allow the blower to draw 100% ambient air into the envelope. Warm air inside the envelope is vented to atmosphere via duct 31 by operation of valve 25, so that the contents of the envelope returns to ambient temperature. The treated article can then be removed.

The treatment has been found to be very effective at denaturing many of the allergenic proteins present in dust mite droppings and other similar materials, so that they no longer produce allergic reactions in susceptible individuals. It should however be appreciated that there are many different allergenic proteins and some will be 100% denatured by the treatment described above whereas other may be denatured to a lesser extent. More effective treatment can generally be achieved by increasing the temperature and the heating period.

Since the treatment kills the dust mites themselves, the production of further allergenic material is prevented for a significant period. Generally, repetition of the treatment at intervals of about 52 weeks will ensure that the allergens and mites are kept at sufficiently low levels to prevent allergic reactions in most people.

Although the above example refers specifically to the treatment of dust mites it will be appreciated that a similar heat treatment regime can be used to kill other pests such as fleas, viruses or bacteria. The treatment conditions might of course have to be adjusted accordingly, e.g. by increasing the temperature and/or the period of heat treatment.

### Drying

The apparatus can be used to achieve controlled drying of items of furniture etc. which might have become wet through flood damage for example. For this purpose the apparatus can conveniently be modified by interposing a dehumidifier unit 50 between the envelope 2 and the machine 12 as shown in FIG. 4. It will however be appreciated that the dehumidifier could be integrated into the machine 12 if desired. The dehumidifier could take various forms. For example, drying could be achieved by passing the moisture-containing air through a rotating wheel containing desiccant crystals. In the apparatus shown in FIG. 4 however, the dehumidifier includes a compressor 51 which is controlled by the microprocessor 22. Connection of the compressor to the unit 12 is automatically detected by the microprocessor 22. The compressor circulates refrigerant through heating coils 52 which are arranged to heat air passing through the duct 30, and condensing coils 53 which cool air returning via the duct 31. Thus the heated air entering the envelope 2 assists evaporation of moisture in an article placed in the envelope 2 whilst the moisture is condensed out by the coils 53 before being circulated through the unit 12.

In this case the unit 12 acts mainly to control the compressor 51 and recirculate air by means of the blower 16. The humidity within the envelope 2 is monitored by the sensor 41 so that the equipment automatically shuts off when all the moisture in the article has been removed.

Water condensing on the coils 53 collects in a tray 54 which is periodically emptied by a pump 55.

### Chemical Impregnation

The apparatus can be used to treat an article with chemicals such as flame retardants in liquid and/or gaseous form. The chemicals are placed in the reservoir 19 and are introduced into the recirculating air, with vaporisation taking place in the heater box 17 if necessary.

Also articles containing wood or wood products can be treated with preservative or other chemicals using the apparatus.

A particularly interesting use of the apparatus lies in the application of borate treatment for wood preservation. For this purpose a slight negative pressure must be maintained in the envelope 2 relative to atmosphere. This is achieved by placing a supporting framework inside the envelope and reversing the blower 16. A supply of trimethyl borate is placed in the reservoir 19 which is then introduced into the recirculating air supply to the envelope 2. The negative pressure caused the chemical to pass into the article contained in the envelope 2, where it hydrolyses with moisture in wood to form boric acid, which acts as a preservative and fire retardant.

Clearly, other wood treatments can be applied in a similar manner.

It will furthermore be appreciated that by making design changes to the apparatus within the scope of the present invention, the operating temperature can be increased sufficiently to permit the apparatus to be used for carrying out other industrial processes.

It will be appreciated that the features disclosed herein may be present in any feasible combination. Whilst the above description lays emphasis on those areas which, in combination, are believed to be new, protection is claimed for any inventive combination of the features disclosed herein.

I claim:

1. Apparatus for treating an article, which includes:
a flexible inflatable envelope for receiving said article;
an inflation unit comprising blower means and having an external air input through which atmospheric air may be drawn for inflating said envelope;
a first air-conducting passage leading from said inflation unit to said envelope; and
a second passage leading from said envelope to said inflation unit whereby air from the envelope may be recirculated through the inflation unit.

2. Apparatus according to claim 1, in which the inflation unit comprises heating means for heating air supplied to the envelope.

3. Apparatus according to claim 1, which includes a fluid reservoir containing a liquid or a gas and means for introducing said fluid into the air flow to the envelope.

4. Apparatus according to claim 3, in which the inflation unit comprises heating means for heating air supplied to the envelope and the fluid is a liquid which is introduced into the heating means such that the liquid is vaporised before being supplied to the envelope.

5. Apparatus according to claim 1, in which the cross sectional area of the first passage is greater than that of the second passage.

6. Apparatus according to claim 1, in which a minor proportion of external air is added to the air which is recirculated through the inflation unit.

7. A method of treating an article, which includes:
placing said article in a flexible envelope;
inflating said envelope with external atmospheric air by means of an inflation unit which feeds the air to the envelope through a second passage; and
recirculating air from the envelope through the inflation unit by returning air to said inflation unit through a first air-conducting passage.

8. A method according to claim 7, which includes heating recirculated air during passage through the inflation unit.

9. A method according to claim 7, in which the inflation unit introduces a liquid or a gas into the air flow to the envelope.

10. A method according to claim 9, in which the fluid is a liquid which is introduced into heating means for heating air passing through the inflation unit such that the liquid is vaporised before being supplied to the envelope.

11. A method according to claim 7, in which a minor proportion of external air is added to the air which is recirculated through the inflation unit.
